(19) United States
(12) Patent Application Publication
(10) Pub. No.: US 2021/0196592 A1
(43) Pub. Date: Jul. 1, 2021

(54) ANTIPERSPIRANT COSMETIC PRODUCT WITHOUT CYCLIC SILICONE OILS

(71) Applicant: Henkel AG & Co. KGaA, Duesseldorf (DE)

(72) Inventors: INGA KERSTIN VOCKENROTH, Duesseldorf (DE); SANDRA MAUSBERG, Haan (DE); IRIS MARINA STADLER, Duesseldorf (DE)

(73) Assignee: Henkel AG & Co. KGaA, Duesseldorf (DE)

(21) Appl. No.: 17/132,948

(22) Filed: Dec. 23, 2020

(30) Foreign Application Priority Data
Dec. 27, 2019 (DE) 10 2019 135 833.7

(51) Int. Cl. A61K 8/37; A61K 8/31; A61K 8/41; A61K 8/26; A61K 8/891; A61K 8/49; A61Q 15/00

(52) U.S. Cl. CPC A61K 8/37; A61K 8/31; A61K 8/416; A61K 2800/34; A61K 8/891; A61K 8/4973; A61Q 15/00; A61K 8/26

(57) ABSTRACT

The present disclosure concerns an antiperspirant cosmetic composition which provides a good skin feeling without cyclic silicone oils. The antiperspirant cosmetic composition may include ethylhexyl palmitate, isopropyl myristate and/or isopropyl palmitate, and at least one C9-C12 alkane.

### CROSS-REFERENCE TO RELATED APPLICATION

This application claims priority to German Patent Application No. 10 2019 135 833.7, filed Dec. 27, 2019, which is incorporated herein by reference in its entirety.

### TECHNICAL FIELD

The present disclosure concerns an antiperspirant cosmetic product which provides a good skin feel without cyclic silicone oils. Thereby the perception of scent can be prolonged. Furthermore, the present disclosure relates to a cosmetic process for reducing perspiration and/or body odor caused by perspiration by using an antiperspirant cosmetic product as contemplated herein.

### BACKGROUND

Washing, cleaning, and caring for one's own body is a basic human need and modern industry is constantly trying to meet these needs in a variety of ways. Particularly important for daily hygiene is the permanent elimination or at least reduction of body odor and underarm wetness. Numerous special deodorizing or antiperspirant body care products are known in the state of the art, which have been developed for use in body regions with a high density of sweat glands, especially in the armpit area. These are packaged in a wide variety of dosage forms, for example as powder, in stick form, as aerosol spray, pump spray, liquid and gel roll-on application, cream, gel and as impregnated flexible substrates (deodorants).

State-of-the-art cosmetic antiperspirants contain, in addition to at least one oil or wax and a fragrance component or perfume, at least one antiperspirant compound, for example in the form of aluminum salts and/or aluminum-zirconium salts, in particular in the form of halides and/or hydroxyhalides of aluminum and/or zirconium.

Antiperspirants and deodorants usually contain silicone oils to create a good feeling on the skin. Decamethylcyclopentasiloxane is frequently used. Furthermore, corresponding silicone oils have the advantage that they are volatile so that they do not leave stains or smear films on the user's clothing. However, these cyclic silicone oils are increasingly criticized because they are not or only with difficulty degradable.

There is therefore a need for antiperspirants and deodorants that do not pollute the environment but at the same time perform well and leave a good feeling on the skin. In addition, the application of the product to the skin should not contaminate clothing. The product should also leave a gentle skin feeling and be absorbed quickly. Furthermore, the product should have a long fragrance and not cause intolerance.

### BRIEF SUMMARY

The present disclosure concerns an antiperspirant cosmetic composition which provides a good skin feeling without cyclic silicone oils. An exemplary antiperspirant cosmetic composition includes ethylhexyl palmitate, isopropyl myristate and/or isopropyl palmitate, and at least one C9-C12 alkane.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has been found that cyclic silicone oils can be replaced by a combination of ethylhexyl palmitate, isopropyl myristate and/or isopropyl palmitate and a natural oil in cosmetic products to reduce sweat and/or body odor caused by sweat. In a first embodiment, therefore, the task underlying the present disclosure is solved comprehensively by an antiperspirant cosmetic composition comprising:

(a) Ethylhexyl palmitate,
(b) Isopropyl myristate and/or isopropyl palmitate; and
(c) at least one C9-C12 alkane.
Surprisingly, it has been shown that the combination of these ingredients can replace cyclic silicone oils without adverse effects on the product. In addition, it has been shown that an agent or composition as contemplated herein exhibits improved fragrance adhesion than a comparable formulation which, however, contains corresponding cyclic silicone oils, such as decamethylcyclopentasiloxane.

As contemplated herein, the term “antiperspirant” is understood to mean the decrease or reduction of perspiration of the body's sweat glands or the reduction of body odor caused by sweat. An antiperspirant cosmetic product within the meaning of the present disclosure can thus be an antiperspirant or a deodorant. In certain embodiments, it is an antiperspirant.

Unless otherwise indicated, percentages by weight in the present disclosure refer to the total amount of the cosmetic product, excluding any propellant.

Preferably the proportion of ethylhexyl palmitate is from about 12% to about 28% by weight, such as preferably from about 15% to about 25% by weight, based on the total weight of the composition.

The composition as contemplated herein may contain isopropyl myristate and/or isopropyl palmitate. Preferably it contains isopropyl myristate. The proportion of isopropyl myristate and/or isopropyl palmitate is preferably from about 4% to about 9% by weight, from about 5% to about 8% by weight, based on the total weight of the composition. The quantities stated refer to the total amount of isopropyl myristate and isopropyl palmitate if both components are present. If the composition contains only one of the two components, the quantity specifications refer accordingly to the one component.

As a further component, the present disclosure contains at least one C9-C12 alkane. Appropriate alkanes can be added in the form of a natural oil, for example. Suitable oils are commercially available, for example, from the company Sasol (Johannesburg, South Africa) under the product name Parafol®. The composition as contemplated herein contains the at least one C9-C12 alkane preferably in a proportion of from about 2 wt. % to about 18 wt. %, of from about 5 wt. % to about 13 wt. %, based on the total weight of the composition.

The cosmetic product as contemplated herein is particularly preferred to be free of cyclic silicone oils, especially free of decamethylcyclopentasiloxane.

The cosmetic product as contemplated herein still preferably contains one or more antiperspirants. Corresponding antiperspirants are described in the state of the art. The at least one antiperspirant may be in the form of aluminum salts and/or aluminum-zirconium salts, in the form of halides and/or hydroxyhalides of aluminum and/or zirconium. In the context of the present disclosure, the term “halides and/or hydroxyhalides of aluminum and/or zirconium” means in particular chlorides, bromides and iodides of aluminum and zirconium as well as compounds of the formulae Al(OH)yX and Zr(OH)zX, where X in the aforementioned formulae stands for a halide ion. These are, especially chlorides.

However, suitable antiperspirants are also poly(vinylamine-vinylformamide) copolymers as described in WO 2019/166140 A1. WO 2006/018073 A1 reveals various polyamines, including polyethyleneimines, as alternatives to halides and/or hydroxyhalides of aluminum and/or zirconium as antiperspirants. Polyphosphazenes and cyclic amines, which can form an anion, are also described as suitable antiperspirant agents in the state of the art. These and other antiperspirants described in the state of the art can be used as inventive step.

The agent as contemplated herein can be in the form of a suspension, for example. To stabilize the suspension, the agent of the present disclosure may contain components that prevent or reduce sedimentation, i.e. can improve the stability of the suspension. One, two, three or more suitable thickeners can be added for this purpose.

As contemplated herein, the antiperspirant cosmetic product may also contain a cosmetic carrier. The carrier can be aqueous, non-aqueous or alcoholic. There may also be mixtures on different carriers.

In a preferred form, the carrier contains polar esters of lactic acid, citric acid, or carbonic acid with C1-C4 alcohols and their anhydrides. Especially preferred esters are triethyl citrate or propylene carbonate. The ester is preferably contained in a total amount of from 0 wt. % to about 15 wt. %, preferably from about 1 wt. % to about 12 wt. %, particularly preferably from about 5 wt. % to about 10 wt. %, each based on the total weight of the cosmetic product.

Depending on the desired application and form of application, the inventive step may comprise further components, in particular auxiliary substances. These are preferably those selected from the group of solvents, perfume oils, emulsifiers, solubilizers, cosmetic oils, consistency enhancers, propellants, pH-adjusting agents, surfactants, chelating agents, skin cooling agents, skin caring agents, waxes, preservatives, plant extracts, absorbers and mixtures of these additives.

In a preferred form, the agent of the present disclosure is in the form of a sprayable suspension which can be sprayed with a propellant. In this case, the blowing agent is preferably contained in an amount of from about 1% to about 98% by weight, preferably from about 30% to about 95% by weight, preferably from about 40% to about 90% by weight, in particular from about 50% to about 85% by weight, based on the total weight of the cosmetic product. The total weight of the agent is the sum of the weight of all components of the agent including the propellant. Preferred blowing agents (propellants) are propane, propene, n-butane, iso-butane, iso-butene, n-pentane, pentene, iso-pentane, iso-pentene, methane, ethane, dimethyl ether, nitrogen, air, oxygen, Nitrous oxide, 1,1,1,3-tetrafluoroethane, heptafluoropropane, perfluoroethane, monochlorodifluoromethane, 1,1-difluoroethane, tetrafluoropropenes, both individually and in mixtures. Hydrophilic propellants, such as carbon dioxide, can also be used advantageously in the sense of the present disclosure if the proportion of hydrophilic gases is chosen to be low and lipophilic propellant (e.g. propane/butane) is present in excess. Propane, n-butane, iso-butane, and mixtures of these propellants are particularly preferred. It has been shown that the use of n-butane as the only propellant gas can be particularly preferred as contemplated herein.

However, as contemplated herein, it may be equally preferred if the cosmetic product is sprayed as propellant-free pump spray or squeeze spray or applied as roll-on.

The means as contemplated herein may further contain at least one oil component, preferably in an amount of from about 0.05% to about 50% by weight, based on the total weight of the means as contemplated herein. The oil component is preferably one selected from the group of (i) volatile silicone oils, especially volatile linear silicone oils; (ii) volatile non-silicone oils, especially liquid kerosene oils, isoparaffin oils, ester oils and organic alcohols; (iii) non-volatile silicone oils; (iv) non-volatile non-silicone oils; and (v) mixtures thereof. If a volatile or non-volatile silicone oil is contained as an oil component in the agent of the present disclosure, it is a non-cyclic silicone oil. Preferably the oil component is a volatile or non-volatile cosmetic oil selected from the group including non-cyclic silicone oils and non-silicone oils. Volatile cosmetic oils are those which at about 20° C. and 1013 hPa have a vapor pressure of from about 2.66 to about 40,000 Pa, preferably from about 10 to about 12,000 Pa, more preferably from about 13 to about 3,000 Pa and especially from about 15 to about 500 Pa. Non-volatile cosmetic oils are those which have a vapor pressure of less than about 2.66 Pa at about 20° C. and 1013 hPa. As contemplated herein, the preferred silicone oils are those selected from linear polyalkylsiloxanes with a kinematic viscosity at about 25° C. of from about 5 cSt to about 2000 cSt, preferably from about 10 to about 350 cSt and especially from about 5 to about 100 cSt. Suitable compounds are available under the trade name Dow Corning® 200. Other oils preferred by the present disclosure are selected from the esters of dimers of unsaturated C12-C22 fatty acids with monohydric linear, branched, or cyclic C2-C8 alcohols or with polyhydric linear or branched C2-C6 alcohols. As contemplated herein, it may be preferable to use mixtures of volatile silicone oils and volatile non-silicone oils as this gives a drier skin feel. Furthermore, within the scope of the present disclosure, it may be preferred to use a non-volatile silicone oil and/or a non-volatile non-silicone oil to mask insoluble components, such as aluminum salt or talcum, or ingredients dried on the skin.

The use of blends of non-volatile and volatile cosmetic oils is particularly preferred, as this allows parameters such as skin feel, visibility of the residue and stability of the antiperspirant cosmetic product to be adjusted, thus better adapting the product to the needs of the consumer.

The agent as contemplated herein not only has an antiperspirant effect but can also be used to cover odors. To this end, the product shall preferably continue to contain at least one fragrance. In the context of the present disclosure, “perfume” means substances with a molecular weight of from about 74 to about 300 g/mol, which contain at least one osmophoric group in the molecule and have an odor and/or taste, i.e. which are able to excite the receptors of the hair cells of the olfactory system. Osmophoric groups are groups covalently bound to the molecular skeleton in the form of hydroxy groups, formyl groups, oxo groups, alkoxycarbonyl groups, nitrile groups, azide groups, etc. In the context of the present disclosure, perfume oils, perfumes or perfume oil components which are liquid at about 20° C. and 1013 hPa are understood. The perfume is thereby preferably contained in an amount of from about 0.001% to about 10% by weight, particularly from about 0.005% to about 9% by weight, more preferably from about 0.1% to about 7% by weight, particularly preferably from about 0.2% to about 6% by weight and particularly from about 0.2% to about 2% by weight in the means as contemplated herein, in each case based on the total weight of the means as contemplated herein.

To further support the antiperspirant and odor-reducing effect of the inventive step, the inventive step also contains one or more deodorant active ingredients in a preferred form. As contemplated herein, the use of certain deodorant active ingredients has proven to be particularly advantageous. To further support the antiperspirant and odor-reducing effect of the inventive step, the inventive step also contains one or more deodorant active ingredients in a preferred form.

(i) Arylsulfatase inhibitors, ß-glucuronidase inhibitors, aminoacylase inhibitors, esterase inhibitors, lipase inhibitors, lipoxigenase inhibitors and cystathionine ß-lyase inhibitors.
(ii) α monoalkyl glycerol ethers with a branched or linear saturated or unsaturated, optionally hydroxylated C6-C22 alkyl radical, α-(2-ethylhexyl)glycerol ether,
(iii) Alcohols, especially phenoxyethanol, benzylheptanol, 1,2-hexanediol, 1,2-octanediol, 1,2-decanediol, tropolone and butyloctanoic acid.
(iv) antibacterial perfume oils.
(v) prebiotically effective components.
(vi) Trialkyl citric acid esters, especially triethyl citrate.
(vii) Active ingredients that reduce the number of skin germs from the group of staphylococci, coryne bacteria, anaerocococci and micrococci involved in the formation of odors or inhibit their growth.
(viii) Zinc and silver compounds, especially zinc phenolsulfonate, zinc cicinoleate, bischloride(1)-octahydroxy-pentazinc, zinc citrate and silver lactate:
(ix) Organohalogen compounds, especially triclosan, chlorhexidine and chlorhexidine gluconate.
(x) Benzalkonium halides, especially benzalkonium chloride and benzethonium chloride.
(xi) quaternary ammonium compounds, especially cetylpyridinium chloride.
(xii) antimicrobially active carbonates, phosphates, and sulfates, especially sodium bicarbonate, cocamidopropyl PG-dimonium chloride phosphates, disodium pyrophosphate and soya morpholinium ethosulphates.
(xiii) Lantibiotics.
(xiv) Aluminum and aluminum-zirconium salts.
(xv) Bispyridines, special Octenidine.
(xvi) antimicrobially active acids, especially caprylic hydroxamic acid, carnesolic acid, tartaric acid and 2-butyloctanoic acid.
(xvii) Polyglycerol esters, especially polyglyceryl-3 caprylates.
(xviii) Sorbitan esters and lactones, especially sorbitan caprylate and glucono-1,5-lactone.
(xix) and mixtures thereof, phenoxyethanol and/or caprylylglycol and/or 1,2-hexanediol and/or α-(2-ethylhexyl)glycerol ether and/or 2-butyloctanoic acid and/or triethyl citrate.

The use of these active ingredients leads not only to a high and long-lasting perfume and a high storage stability, but also to a high and long-lasting deodorizing effect.

Depending on the intended use, the inventive step can be applied in various ways, for example in the form of a spray, a stick, a soft solid, a cream, a gel, or a roll-on. In some cases, it has also proved to be advantageous to apply the inventive step on or in a disposable substrate, for example in the form of cloths, pads, or puffs.

Depending on the intended use, the cosmetic product as contemplated herein may therefore be in solid, semi-solid, liquid, disperse, emulsified, suspended, gel, multi-phase, or powder form. For the purposes of the present disclosure, the term liquid also includes any kind of solid dispersions in liquids. Furthermore, multi-phase cosmetic products according to the present disclosure are defined as products which have at least two different phases with a phase separation and in which the phases can be arranged horizontally, i.e. one above the other, or vertically, i.e. next to each other. The application can be done for example with an aerosol spray, a roller ball applicator, a pump sprayer or with a fixed pen.

Another object of the present disclosure is a non-therapeutic cosmetic method for preventing and/or reducing body odor and/or perspiration of the body, in which a cosmetic agent is applied to the skin, in particular to the skin of the armpits, as described above and remains on the skin of the armpits for at least 1 hour, preferably for at least 2 hours, preferably for at least 4 hours, in particular for at least 6 hours.

Preferred embodiments of the present disclosure may be combined in any way, regardless of the embodiment or subject matter of the present disclosure. Thus, the preferred forms of execution described for the product apply accordingly for its use.

In the following, the present disclosure will be explained based on design examples without, however, restricting it.

### EXAMPLES

All figures are given in weight percent, whereby the quantities refer to the commercially available product used (without any propellants).

1. Wash Test for Anti-Stain Performance

The following compositions (E: inventive, V: comparative formulation) manufactured and tested:

| Component (INCI name) | E1 | V1 | V2 |
|---|---|---|---|
| Ethylhexyl Palmitates | 20.00 | 30.00 | 30.00 |
| Isopropyl Myristates | 6.67 | 10.00 | 6.67 |
| Perfume | 6.57 | 6.57 | 6.57 |
| Disteardimonium Hectorite | 2.53 | 2.53 | 2.53 |
| Aluminum Chlorohydrate | 25.53 | 25.53 | 25.53 |
| Dimethicone | 10.00 | 10.00 | 10.00 |
| Propylene Carbonate | 5.00 | 5.00 | 5.00 |
| C9-12 Alkanes | 10.00 | 7.00 | 10.14 |
| Triethyl Citrate | 10.00 | 3.37 | 3.56 |
| C12-15 Alkyl Benzoate | 3.70 | — | — |

The following materials were used for the tests:
blue woven cotton fabric (light blue, woven 100% cotton polo jersey), test sample size: 15×15 cm
white knitted 100% cotton material, test sample size: 15×15 cm

The aerosol products were applied to these samples for 2 seconds from 15 cm. Then 1 ml of artificial sweat was added. After the samples lay overnight, they were washed the following day in a washing machine (Miele Softtronic w 1714):

| Washing machines | White fabric | Blue fabric |
|---|---|---|
| Loading | 3.5 kg fill wash | 3.5 kg fill wash |
| Water quantity | 17 liters | |
| Washing temperature | 40° C. | |
| Quantity of detergent | 80 g Persil Universal | 70 g Spee |
| | Powder Gold | Color Gel |

The samples were then dried in a tumble dryer (Miele Novotronic T 7644C), then ironed and subjected to visual assessment. The process was then repeated a total of 8 times. After 14 days of storage in the dark, another assessment was made in comparison to a reference scale (0=no contamination, 4=very heavy contamination). The result is the mean value of the evaluation after storage:

| | E1 | V1 | V2 |
|---|---|---|---|
| White Spots | 1 | 0.5 | 0.5 |
| Greasy stains | 0 | 1.2 | 1.2 |

2. Cosmetic Parameters in the Consumer Test

The following agents were produced (E: according to present disclosure, V: Comparative formulation) and tested:

| Component (INCI name) | E1 | V3 |
|---|---|---|
| Cyclomethcione | — | 16.40 |
| Ethylhexyl Palmitates | 20.00 | 30.0 |
| Isopropyl Myristates | 6.67 | 6.00 |
| Perfume | 6.57 | 6.67 |
| Disteardimonium Hectorite | 2.53 | 2.53 |
| Aluminum Chlorohydrates | 25.53 | 23.53 |
| Dimethicone | 10.00 | 10.00 |
| Propylene Carbonate | 5.00 | 0.87 |
| C9-12 Alkanes | 10.00 | — |
| Triethyl Citrate | 10.00 | 4.00 |
| C12-15 Alkyl Benzoate | 3.70 | — |

20 test persons used the products under consumer conditions for 1 week at home. Subsequently, you evaluated both products about different properties:

| | E1 | V3 | both equally | both equally |
|---|---|---|---|---|
| 20 test persons | better | better | good | bad |
| Skin feeling after | 6 | 2 | 12 | — |
| application | | | | |
| Duration of scent | 5 | 2 | 11 | 2 |
| perception | | | | |
| Minimal soiling of the | 3 | — | 16 | — |
| laundry | | | | |

Although the product as contemplated herein is free of cyclic siloxanes, which are used to improve skin feel, the test persons found the product as contemplated herein to be better. The duration of the scent perception as well as the low soiling of the laundry was considered by the test persons as improved compared to the reference product.

Thus, the product as contemplated herein not only enables an equivalent replacement for cyclic silicone oils in terms of skin feel, but also leads to an improvement in terms of the duration of fragrance perception

3. Skin Compatibility Study by Patch Test

The following agents were produced (E: according to present disclosure, V: Comparative formulation) and tested:

| Component (INCI name) | E1 | V4 |
|---|---|---|
| Ethylhexyl Palmitates | 20.00 | 7.70 |
| Isopropyl Myristates | 6.67 | 6.67 |
| Perfume | 6.57 | 6.57 |
| Disteardimonium Hectorite | 2.53 | 2.53 |
| Aluminum Chlorohydrates | 25.53 | 25.53 |
| Dimethicone | 10.00 | 10.00 |
| Propylene Carbonate | 5.00 | 5.00 |
| C9-12 Alkanes | 10.00 | 20.00 |
| Triethyl Citrate | 10.00 | 12.00 |
| C12-15 Alkyl Benzoate | 3.70 | 4.00 |

On the back of 16 test persons 70 μl of the manufactured solutions were applied on each side next to the spine. 0.5% SDS (Sodium Dodecyl Sulfate) and 1% Texapon N70 solution were used as positive samples, demineralized water, and cosmetic alcohol as negative samples. After 5 minutes, the treated areas were covered with occlusive non-adsorbing foil. The next day the materials were removed after 24 hours. 6 hours after removal, the body areas were analyzed by trained personnel. A further evaluation was performed 24 h, 48 h, and 72 h after removal of the foils.

The degree of irritation was calculated in % to SDS. For this purpose, the number of skin irritations with SDS was set to 100% and the other products were based on this. The test was considered passed for E1 and confirmed good cosmetic compatibility of the product, whereas V4 did not pass.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An antiperspirant cosmetic composition comprising:
(a) Ethylhexyl palmitate;
(b) isopropyl myristate and/or isopropyl palmitate; and
(c) at least one C9-C12 alkane.

2. The composition according to claim 1, wherein the proportion of ethylhexyl palmitate is from about 12% to about 28% by weight, based on the total weight of the composition.

3. The composition according to claim 1, wherein it comprises isopropyl myristate and/or isopropyl palmitate in a proportion of from about 4% to about 9% by weight, based on the total weight of the composition.

4. The composition according to claim 1, wherein it comprises the at least one C9-C12 alkane in a proportion of from about 2% to about 18% by weight, based on the total weight of the composition.

5. The composition according to claim 1, wherein the composition is free from cyclic silicone oils.

6. The composition according to claim 1, wherein the agent further comprises at least one perfume.

7. The composition according to claim 1, wherein it further comprises at least one antiperspirant.

8. The composition according to claim 1, wherein the composition comprises:
(a) Ethylhexyl palmitate;
(b) Isopropyl myristate; and
(c) at least one C9-C12 alkane;
and wherein the composition is free of cyclic silicone oils.

9. A non-therapeutic cosmetic method for preventing and/or reducing body odor and/or perspiration of the body, in which the antiperspirant cosmetic composition according to claim 1 is applied to the skin and remains on the skin for at least about 1 hour.

10. The composition according to claim 1, wherein:
the proportion of ethylhexyl palmitate is from about 15% to about 25% by weight, based on the total weight of the composition;
the composition comprises isopropyl myristate and/or isopropyl palmitate in a proportion of from about 5% to about 8% by weight, based on the total weight of the agent;
the composition comprises the at least one C9-C12 alkane in a proportion of from about 5% to about 13% by weight, based on the total weight of the agent;
the composition is free of decamethylcyclopentasiloxane.

11. The composition according to claim 10, further comprising:
at least one perfume; and
at least one antiperspirant.

12. The composition according to claim 10, wherein the composition comprises (b) isopropyl myristate, and wherein the composition is free of cyclic silicone oils.
